(19) United States
(12) Patent Application Publication
(10) Pub. No.: US 2019/0038422 A1
(43) Pub. Date: Feb. 7, 2019

(54) COBRA TYPE FEMORAL STEM PROSTHESIS

(71) Applicant: Beijing Lidakang Technology Co.,Ltd., BEIJING (CN)

(72) Inventors: GUOQAING ZHANG, BEIJING (CN); NING XU, BEIJING (CN)

(21) Appl. No.: 15/944,787

(22) Filed: Apr. 4, 2018

(30) Foreign Application Priority Data
Aug. 7, 2017 (CN) 201720976081.8
Dec. 6, 2017 (CN) 201721684843.3

(51) Int. Cl. A61F 2/36; A61F 2/30

(52) U.S. Cl. CPC A61F 2/3662; A61F 2/30771; A61F 2/3609; A61F 2002/30205; A61F 2002/3652; A61F 2002/30125; A61F 2310/00407; A61F 2002/30838; A61F 2002/30827

(57) ABSTRACT

The present disclosure provides a cobra type femoral stem prosthesis, that is a femoral stem prosthesis for congenital dysplasia of the hip (CDH) which includes a femoral stem cone, a femoral neck and a stem body which are connected sequentially; the stem body includes proximal segment of femoral stem, middle segment of femoral stem and distal segment of femoral stem, wherein the femoral stem cone is a cone shape; One end of the femoral neck is connected with the proximal segment of femoral stem and there is a deflection angle therebetween and the other end of the femoral neck is connected with the large diameter end of the femoral stem cone.

### CROSS-REFERENCE TO RELATED APPLICATIONS

This application claims priority to Chinese Patent Application No. 201720976081.8 with a filing date of Aug. 7, 2017 and No, 20172168484.3 with a filing date of Dec. 6, 2017, The content of the aforementioned applications, including any intervening amendments thereto, are incorporated herein by reference.

### TECHNICAL FIELD

The present disclosure relates to the technical field of prosthetic replacement, in particular to a cobra type femoral stem prosthesis with a distal segment of fernoral stem having a protruding longitudinal lateral ridge structure.

### BACKGROUND OF THE PRESENT INVENTION

Congenital dysplasia of the hip (CDH) is a disease of abnormal hip joint. The anteversion angle for the patient is significantly higher than that of a normal person, if a doctor fails to select a suitable prosthesis according to the anteversion angle for the patient, it would lead to a series of problems such as a adversely placed prosthesis angle, an inappropriate reconstruction for the rotation center, which will greatly increase postoperative prosthesis dislocation. prosthesis abrasion, postoperative joint dysfunction and other complications, and seriously affect the effect of total hip arthroplasty. Meanwhile, the range of hip joint activity is reduced, a collision of the femoral stem and acetabular components may be occurred easily, and the success rate of the operation is reduced.

The current prosthesis includes modular femoral stem prosthesis and S-ROM prosthesis. The modular femoral stem prosthesis can not achieve an ideal match with the femoral medullary cavity of the Chinese people. The stability of the modular femoral stem prosthesis is poor and the long-term survival rate is low The modular femoral stem prosthesis is difficult to implant and difficult to restoration, and more bone mass needs to used. A assembled junction of S-ROM prosthesis increases the risk of prosthesis fracture and may produce metal debris, which may affect the service life of prosthesis. Modular femoral stem prosthesis and S-ROM prosthesis both have such problems as abrasion between components and osteolysis, reducing the biological compatibility of the prosthesis, making a large inventory of components to be borne, and unfavourable to small-scale manufacturers. Modular femoral stem prosthesis and S-ROM prosthesis both need to be configured according to a geometric shape of femoral medullary cavity and anteversion angle, for the patient. The configuration during surgery will prolong the, operative time, which may increase the suffering of patients, and reduce the success rate of operation.

### SUMMARY OF PRESENT INVENTION

The technical problem to be solved by the disclosure is to provide a cobra type femoral stem prosthesis, in order to solve the problems of current prosthesis such as abrasion between components, osteolysis, poor biocompatibility of the prosthesis, large inventory, high cost, configuration of the prosthesis during surgery, long time operation, increase of the sufferings of patients and low success rate of the operation.

In order to solve the technical problems, the embodiment of the disclosure provides a cobra type femoral stem prosthesis, wherein the femoral stem prosthesis for congenital dysplasia of the hip (CDH) includes a femoral stem cone, a femoral neck and a stem body which are connected sequentially; the stem body includes proximal segment of femoral stem and middle segment of femoral stem and distal segment of femoral stem, characterized in that, the femoral stem cone is a cone shape; one end of the femoral neck is connected with the proximal segment of femoral stem and there is a deflection angle therebetween, and the other end of the femoral neck is connected with the large diameter end of the femoral stem cone; the surface of the proximal segment of femoral stem is provided with a plurality of annular microgrooves, and the outer surface of the distal segment of femoral stem is provided with a plurality of longitudinal lateral ridge extending longitudinally.

Wherein, there is an acute angle between the axis of the proximal segment of femoral stem and the axis of the femoral neck, which is 0°˜35° or 145°˜180°.

Wherein, the angle between the central line of the stem body and the central line of the femoral neck is 120°˜140°.

Wherein, the outer surface of the distal segment of femoral stem is provided with four to ten lateral ridges, which have a height of 0.55˜0.65 mm.

Wherein, the cross section of the femoral neck is oval

Wherein, the annular microgrooves have a width of 1.0˜1.5 mm, and have a depth of 0.4˜0.5 mm.

Wherein, the outer surface of the proximal segment of femoral stem is sprayed with Ti coating, which has a thickness of 50˜800 μm.

The beneficial effects of the technical scheme of the disclosure are as follows:

In the above scheme, the prosthesis adopted in the disclosure is integrated without any need to assemble components, eliminating problems such as abrasion between components and osteolysis, reducing inventory, leading to low cost, and suitable for all kinds of large-scale or small-scale factory; the design of the femoral stem of the disclosure conforms to the femoral stem for geometric shape of femoral medullary cavity of more than 90% patients; the anteversion angle in the disclosure is 0°˜35° or 145°˜180°, and patients for surgery can choose appropriate specifications of prosthesis according to their own anteversion angle, which effectively shorten the operation time, reduce the pain of patients, and improve the success rate of the operation; Ti coating is sprayed on the outer surface of the proximal segment of the femoral stem in the disclosure, enhancing, biological compatibility of the prosthesis, and the strength and ductility and toughness match with human bone, which creates favorable conditions for bone cells and soft tissue ingrowth and proliferation, so as to achieve the perfect matching of the prosthesis with the patient's femora, especially suitable for developmental dysplasia of the hip (DDH) patients with congenital dislocation of the hip (CDH).

### DESCRIPTION OF THE DRAWINGS

FIG. 1 is a schematic diagram of the cobra type femoral stem prosthesis according to the disclosure;

FIG. 2 is a coronal diagram of the cobra type femoral stem prosthesis according to the disclosure;

FIG. 3 is a diagram of a deflection angle of the artificial alpha femoral stem prosthesis according to the disclosure.

### DESCRIPTION OF MAIN COMPONENT SIGN

1—femoral stem cone;

2—femoral neck:

3—stem body;

4—proximal segment of the femoral stem;

5—middle segment of the femoral stem;

6—distal segment of the femoral stem;

7—annular microgrooves;

8—longitudinal lateral ridge

### DETAILED DESCRIPTION OF PREFERRED EMBODIMENTS

In order to make the technical problems, technical solutions and advantages to be solved more clearly, the following drawings and specific examples will be described in detail.

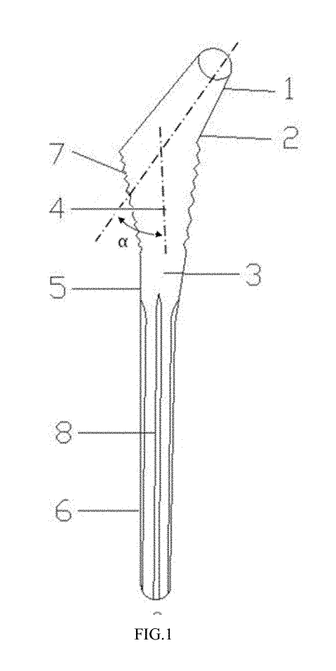

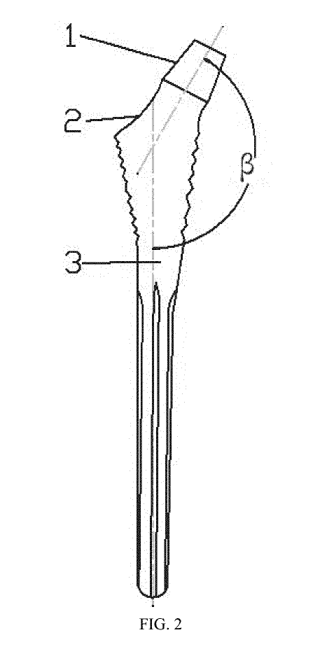

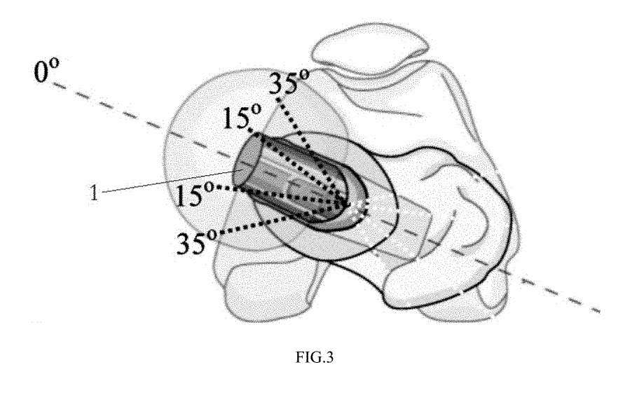

As shown in FIG. 1 to FIG. 3, a cobra type femoral stem prosthesis of the disclosure includes a femoral stem cone 1, a femoral neck 2 and a stem body 3 which are connected sequentially; wherein the stem body includes a proximal segment of femoral stem 4, a middle segment of the femoral stem 5 and a distal segment of the femoral stem 6. The femoral stem cone 1 is a cone shape. There is a deflection angle between one end of the femoral neck 2 and the proximal segment of femoral stem 4, and the other end of the femoral neck 2 is connected with the large diameter end of the femoral stem cone 1.

The outer surface of the proximal segment of the femoral stem is provided with a plurality of annular microgrooves 7, which effectively increases the contact area of the prosthesis and the bone interface, and ensures the stability of the prosthesis after the prosthesis is implanted. In the present embodiment, sawtooth groove is optimally selected for the annular microgroove, with a groove width of 1.0˜1.5 mm and a groove depth of 0.4˜0.5 mm.

Wherein, the angle between the axis of the proximal segment of the femoral stem 4 and the axis of the femoral neck 2 is angle α, which is anteversion angle and is 0°˜35° [symbol] 145°˜180°, preferably 15°, 35°, 145° and 165°.

Furthermore, the angle between the central line of the stem body 3 and the central line of the femoral neck 2 is 120°˜140°, and the optimum value is 130°. The design of this angle is more in line with the structure of human physiological anatomy.

The outer surface of the distal segment of the femoral stem is provided with four to ten longitudinal lateral ridge 8, preferably eight, enhancing the initial stability of the prosthesis and antirotation performance. In this embodiment, the height of the longitudinal lateral ridge is 0.55˜0.65 mm, that is the outside diameter of the longitudinal lateral ridge is about 1.25 mm more than the foundation diameter of the femoral stem.

Wherein, the cross section of the femoral neck 2 is oval, which increases the range of activity of the prosthesis and reduces the abrasion between the femoral neck 2 and the lining of the total acetabulum.

Wherein, Ti coating is sprayed on the surface of the proximal segment of the femoral stem 4, wherein the thickness of the coating is 50˜00 um, the porosity factor is 10˜80%, and the bonding strength of the coating is more than or equal to 25 MPa.

The prosthesis adopted in the disclosure is integrated, without any need to assemble components, eliminating problems such as abrasion between components and osteolysis, reducing inventory, leading to low cost, suitable for all kinds of large-scale or small-scale factory; the design of the femoral stem of the disclosure conforms to the femoral stem for geometric shape of femoral medullary cavity of more than 90% patients; the anteversion angle in the disclosure is °˜35° or 145°˜180°, and patients for surgery can choose appropriate specifications of prosthesis according to their own anteversion angle, which effectively shorten the operation time, reduce the pain of patients, and improve the success rate of the operation; Ti coating is sprayed on the outer surface of the proximal segment of the femoral stem 4 in the disclosure, enhancing biological compatibility of the prosthesis, and the strength and ductility and toughness match with human bone, which creates favorable conditions for bone cells and soft tissue ingrowth and proliferation, so as to achieve a perfect match, between the prosthesis and the patient's femora, especially suitable for developmental dysplasia of the hip (DDH patients with'congenital dislocation of the hip (CDH).

The above is the preferred embodiment of the disclosure, it should be pointed out that for those of ordinary skill in the art, some improvement and polishing can be made without departing from the principle of the disclosure, and these improvements should also be regarded as the scope of protection of the disclosure.

We claim:

1. A cobra type femoral stem prosthesis, that is a femoral stem prosthesis for congenital dysplasia of the hip (CDH), which comprises a fernoral stem cone, a femoral neck and a stem body which are connected sequentially; the stem body comprising a proximal segment of a femoral stem, a middle segment of the femoral stem and a distal segment of the femoral stem, characterized in that the femoral stem cone is a cone shape; one end of the femoral neck is connected with the proximal segment of the femoral stem and there is a deflection angle therebetween, and the other end of the femoral neck is connected with large diameter end of the femoral stem cone; a surface of the proximal segment of the femoral stem is provided with a plurality of annular microgrooves; and an outer surface of the distal segment of the femoral stem is provided with a plurality of longitudinal lateral ridges extending longitudinally.

2. The cobra type femoral stem prosthesis according to claim 1, characterized in that, an acute angle between an axis of the proximal segment of the femoral stem and an axis of the femoral neck is 0°˜35° or 145°˜180°.

3. The cobra type femoral stem prosthesis according to claim 1, characterized in that, an angle between a central line of the stem body and a central line of the femoral neck is 120°˜140°.

4. The cobra type femoral stem prosthesis according to claim 1, characterized in that, the outer surface of the distal segment of the femoral stem is provided with four to ten lateral ridges, which have a height of 0.55˜0.65 mm.

5. The cobra type femoral stem prosthesis according to claim 1, characterized in that, cross section of the femoral neck is oval.

6. The cobra type femoral stem prosthesis according to claim 1, characterized in that, the annular microgrooves have a width of 1.0˜1.5 mm, and have a depth of 0.4˜0.5 mm.

7. The cobra type femoral stem prosthesis according to claim 1, characterized in that, the outer surface of the proximal segment of the femoral stem is sprayed with Ti coating which has a thickness of 50˜800 um.
